(19) United States
(12) Patent Application Publication
(10) Pub. No.: US 2008/0262477 A1
(43) Pub. Date: Oct. 23, 2008

(54) Hand made catheter to be used in suprapubic prostatectomy

(75) Inventor: Hooman Djaladat, Bandar Abbas (IR)

Correspondence Address: JANUS - PATENTS, P.O. BOX # 1345, ARROBEAR, CA, 92382-1500, US

(73) Assignee: Dr. Hooman Djaladat, Bandar Abbas (IR)

(21) Appl. No.: 11/875,880

(22) Filed: Oct. 20, 2007

Related U.S. Application Data
(60) Provisional application No. 60/913,247, filed on Apr. 20, 2007.

(51) Int. Cl. A61M 27/00

(52) U.S. Cl. 604/544

(57) ABSTRACT

A catheter system suitable for use in suprapubic prostatectomy is provided. The catheter system includes a multi-lumen tube having a plurality of lumens, the multi-lumen tube further comprising a plurality of outlet openings positioned on a first lumen, which are used to facilitate passage of fluid out of the urinary bladder, at least an inlet opening positioned on a second lumen, which is used to facilitate passage of fluid in to the urinary bladder, and an expandable balloon connected to the distal end of the multilumen tube. The catheter system is fixed inside the prostatic lodge, wherein the distal end of the catheter system is positioned in the urethra and is blocked to prevent leakage of fluid through the urethra. The expandable balloon, positioned inside the prostatic lodge, applies pressure on the walls of the prostatic capsule and thus helps in controlling bleeding.

### BACKGROUND OF THE INVENTION

The present invention relates to a medical device. More particularly, the present invention relates to a drainage catheter used after performing a suprapubic prostatectomy.

The prostate gland is a chestnut-shaped organ which surrounds the urethra just below the urinary bladder. The prostrate gland is composed of prostate tissues. Prostate tissue consists of fibromuscular tissue anterior to the urethra and glandular tissue posterior to the urethra. Usually, in aging men, a bilateral nodular expansion of prostate tissue in the periurethral region of the prostate, between the fibromuscular tissue and the glandular tissue is observed. Due to this the prostate gland becomes cancerous or fibroid and increases in size. This increase in size is referred to as benign prostatic hyperplasia (BPH). The increase in size of the prostate inside its capsule exerts pressure on the urethra, which passes through the capsule, resulting in obstruction to urine flow, nocturia and incontinence. Hence, prostate gland needs to be removed or operated.

BPH can be treated by means of surgical interventions including transurethral resection of prostate (TURP), open prostatectomy, and transurethral incision of the prostate (TUIP). TURP and TUIP are usually performed in patients with moderate symptom severity and a small to medium prostate. Open prostatectomy is performed in patients who have a large prostate, a damaged bladder or any other serious related problem. Open prostatectomy can be performed by retropubic or suprapubic approach. The suprapubic approach consists of enucleating the hyperplastic adenoma through an extraperitoneal incision of the anterior bladder wall. After suprapubic prostatectomy, a transurethral catheter or a cystostomy catheter is used to provide post-surgical drainage of the bladder.

In case of transurethral catheters, the transurethral catheter is passed through the urethra of a patient to position the distal end of the catheter in the patient's urinary bladder. Although, transurethral catheters maintain urethral patency while providing post-surgical drainage of the urinary bladder, the use of transurethral catheters may cause urinary tract infection (UTI) upon prolonged indwelling duration, and severe bladder irritation. Further, these catheters are uncomfortable and may result in dislodging from the appropriate bladder position. Moreover, the transurethral catheters can also serve as a pathway for pathogens which migrate from the skin surface through the urethra to the anastomotic site and the bladder, thereby leading to sepsis and affecting the healing process. This affects patient's periurethral normal flora, aseptic catheterization and indwelling duration.

In light of the forgoing, what is needed is a catheter system for use in suprapubic prostatectomy with a low postoperative risk of infection, incontinence and stricture formation.

### SUMMARY

An object of the invention is to provide urethral catheter-free suprapubic prostatectomy

Another object of the invention is to provide a catheter system for use in suprapubic prostatectomy having a low postoperative risk of infection, incontinence and stricture.

To achieve the above mentioned objectives, various embodiments of the present invention provide a catheter system suitable for use in suprapubic prostatectomy. The catheter system is used for drainage of fluids from the urinary bladder. The catheter system includes multilumen tube with an expandable balloon at its distal end, at least two outlet openings proximal to the expandable balloon and at least one inlet opening placed in between the at least two outlet openings. The multi-lumen tube extends longitudinally and has at least one drainage passage and an inlet passage. The two outlet openings are placed on the drainage passage and the inlet opening is placed on the inlet passage. The inlet passage is used for transfer of any medicine or drug into the urinary bladder. Further, one of the lumens of the multi-lumen tube is used as an inflation passage to inject fluid into the expandable balloon for its expansion.

The catheter system is fixed inside the prostatic lodge. The distal end of the catheter system, i.e., the tip of the catheter system distal to the expandable balloon is inserted inside the posterior urethra. The opening at the distal of the catheter system is sealed by tying a knot using a silver thread to prevent leakage of any fluid through the urethra. The expandable balloon which is inserted inside the prostatic lodge applies pressure on the walls of the prostatic capsule and thus helps in controlling bleeding. This results in reduced retention of clots and decrease in the lowering of hemoglobin content.

### BRIEF DESCRIPTION OF THE DRAWINGS

The preferred embodiments of the invention will hereinafter be described in conjunction with the appended drawings provided to illustrate the invention.

FIG. 1 illustrates a catheter system suitable for use in suprapubic prostatectomy, in accordance with an embodiment of the present invention;

FIG. 2 illustrates a cross sectional view of the proximal end of a catheter system for use in suprapubic prostatectomy, in accordance with an embodiment of the invention;

FIG. 3 illustrates a catheter system being placed inside the prostatic lodge suprapubically positioned in a patient for drainage of fluid from the urinary bladder, in accordance with an embodiment of the invention; and

FIG. 4 is a flowchart illustrating the surgical technique by using the catheter system, in accordance with an embodiment of the invention.

### DESCRIPTION OF VARIOUS EMBODIMENTS

### Definitions

The following definitions are provided to facilitate understanding of the present invention. The definitions do not limit the scope of the invention:
Catheter: Catheter is a flexible tube which can be inserted in to parts of human body that provides a channel for the passage of fluids. Catheters are deployed in body cavities, ducts or vessels. Catheters are deployed, to drain waste fluids outside the body, to inject fluids inside the body, or to allow the passage of surgical instruments through it.
Prostate gland: The prostate gland is an organ of the male reproductive system. The urethra, a tube which carries urine and semen through the penis, runs across the prostate gland. The location of the prostate gland is between the urinary bladder and the rectum.
Benign prostatic hyperplasia (BPH): BPH is a disease that affects the prostate gland in males. BPH is a noncancerous growth of the prostate gland. The enlargement of the prostate gland exerts pressure on the urethra and results in obstruction of urine flow. This disease commonly occurs in middle-aged and older males.
Transurethral resection of prostate (TURP): TURP is a surgery used to treat BPH. This procedure involves the removal of the enlarged portion of the prostate gland using a cytoscope via the urethral path.
Urinary tract infection (UTI): UTIs are caused due to the invasion and multiplication of bacteria in the urinary system.
Prostatectomy: Prostatectomy refers to the removal of entire or a part of the prostate gland surgically.
Suprapubic prostatectomy: Suprapubic prostatectomy is a transvesical prostatectomy procedure in which incision of the lower anterior urinary bladder wall is made so that direct visualization of bladder mucosa and bladder neck is possible.
Cystostomy: Cystostomy is a surgical procedure used to create an opening in to the urinary bladder.

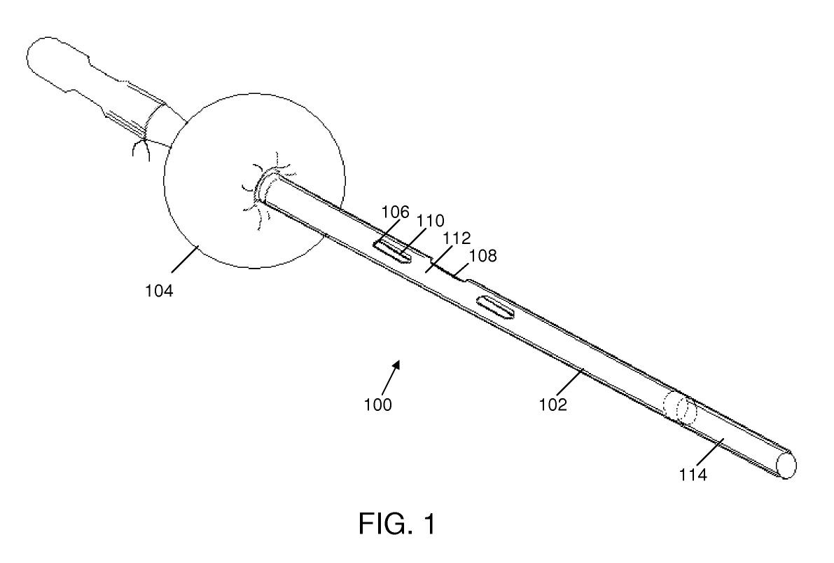

FIG. 1 depicts a catheter system 100 for use in suprapubic prostatectomy in accordance with various embodiments of the invention. Catheter system 100 includes a multi-lumen tube 102 and an expandable balloon 104. Multi-lumen tube 102 includes a plurality of outlet openings 106 and at least one inlet opening 108.

Multi-lumen tube 102 is an elongated tubular structure which extends longitudinally therein. Expandable balloon 104 is attached at the distal end of multi-lumen tube 102. Multi-lumen tube 102 has at least three lumens 110, 112 and 114 used for drainage of fluids and injection of fluids. Outlet openings 106 are present on lumen 110 and are placed proximal to expandable balloon 104. Lumen 110 is also referred to as drainage passage 110. Outlet openings 106 provide external access for urine in the urinary bladder to enter the drainage passage and drain there from into a urine collection bag (not shown in the figure) placed outside the patient's body. Inlet opening 108 is placed on a second lumen 112 and is located between outlet openings 106. Second lumen 112 is also referred to as an inlet passage 112. Fluids such as medicines are injected into the urinary bladder using inlet opening 108. Third lumen 114 is used to inject fluid such as distilled water to inflate expandable balloon 104. Third lumen 114 is also referred to as inflation passage 114. The distal end of catheter system 100, i.e., the portion of multi-lumen tube 102 distal to expandable balloon 104 is closed or sealed and placed in the urethra of a patient after removing the patients prostate adenoma and the proximal end of catheter system 100 is fixed to the bladder mucosa by a 2-zero plain suture next to its entry in to the bladder dome.

In various embodiments of the invention, the distal end of catheter system 100 is imperforate and blocks external egress from drainage passage 110 to minimize urine from contaminating the operated site. Furthermore, the length of the distal portion is sized such that the distal end remains in the fossa navicularis of the urethra. This ensures that the patient's external urethreal orifice is maintained in a collapsed state to minimize the migration of microorganisms or pathogens from accessing the operated site. In an embodiment of the invention the distal end is sealed or closed by tying a 1-0 silk thread.

In an embodiment of the invention, multi-lumen tube 102 is made of a medical silicon grade biocompatible material. In another embodiment of the invention, multi-lumen tube 102 is made of latex or Teflon materials. In addition, to increase fluoroscopic or radiographic visualization of catheter system 100, multi-lumen tube 102 includes a radiopaque material. To minimize the growth of pathogens or microorganisms multi-lumen tube 102 may include a medicant such as antibiotics for example. Trimethoprim or Fluoroquinolons.

Outlet openings 106 and inlet opening 108 are circular in shape. Outlet openings have a cross-section area of about 50 square millimeters and inlet opening has a diameter of about five millimeters. In an embodiment of the invention, the distance between outlet openings 106 is about 10 millimeters. Outlet openings 106 and inlet opening 108 are elliptical in shape. However, any polygonal shape may also be used. Further, in an embodiment of the invention, drainage passage 110 of multi-lumen tube 102 may have only one outlet opening. Further, it will be apparent to those skilled in the art that there can be multiple outlet and inlet openings.

Expandable balloon 104 is made of a biocompatible elastic material such as polytetrafluroethylene (PTFE) or silicon. Expandable balloon 104 in its deflated state is positioned in the prostate lodge and then inflated by injecting fluid such as distilled water through inlet passage 114. Expandable balloon 104 in inflated state exerts pressure on the walls of the prostatic capsule and thus helps in controlling bleeding. In accordance with an embodiment of the invention, catheter system 100 is a 24Fr 3-way Foley catheter system.

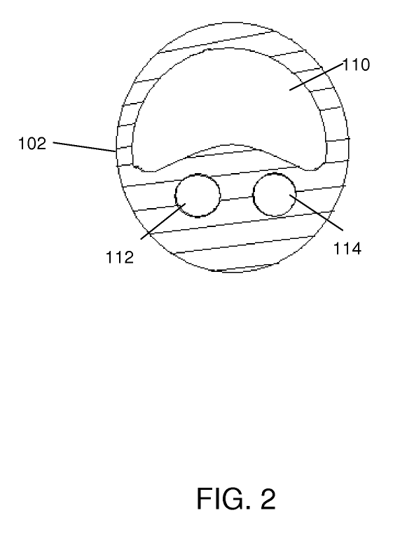

FIG. 2 is a cross sectional view of the proximal end of catheter system 100, in accordance with an embodiment of the invention. FIG. 2 depicts, proximal end of multi-lumen tube 102 including drainage passage 110 and two inlet passages 112 and 114. Drainage passage 110 and inlet passages 112 and 114 are arranged in a side by side configuration or adjacent configuration. In an embodiment of the invention, drainage passage 110 and inlet passages 112 and 114 may be arranged in a coaxial configuration. In various embodiments of the invention, drainage passage 110 has crescent shape and inlet passages 112 and 114 have circular shape. All the three passages extend along the length of multi-lumen tube and are sealed at the distal end placed in the urethra.

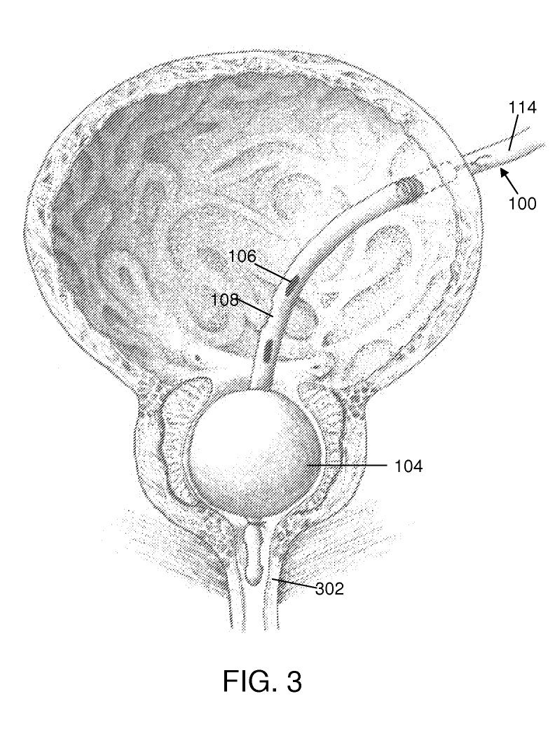

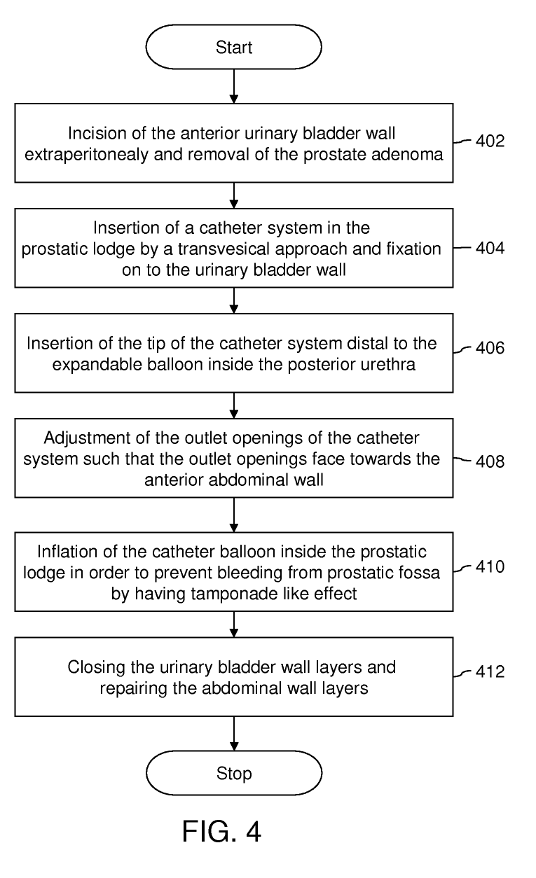

FIG. 3 illustrates a schematic view of catheter system 100 suprapubically fixed in the prostatic lodge by a transvesical approach, for drainage of fluids from the urinary bladder. Catheter system 100 is placed suprapubically during and following excision of the prostate adenoma via open or laparoscopic surgery. The proximal end of catheter system 100 is fixed to the urinary bladder mucosa by a 2-zero plain suture next to its entry in to the urinary bladder dome. Distal portion of catheter system 100 is positioned in urethra 302 through the operated site and is closed by a 1-zero silk. Expandable balloon 104 is positioned in the prostate lodge. Expandable balloon 104 is inflated by injecting distilled water through inlet passage 114. The inflated balloon exerts pressure on the walls of the prostatic capsule and helps in controlling bleeding. Outlet openings 106 and inlet openings 108 are positioned inside the urinary bladder. Urine collected in the urinary bladder enters drainage passage 110 through outlet openings 106. The use of the above described catheter system 100 in a surgical process is described in conjunction with FIG. 4.

FIG. 4 is a flowchart illustrating the surgical technique by using a catheter system, in accordance with an embodiment of the invention. At step 402, the anterior wall of the urinary bladder is cut extraperitonely and the prostate adenoma is removed. At step 404, a catheter system such as catheter system 100 is inserted in the prostatic lodge by a transvesical approach and fixation on to the urinary bladder wall. The catheter system includes a multi-lumen tube such as multi-lumen tube 102 extending longitudinally therein. The multi-lumen tube has three passages such as drainage passage 110, inlet passage 112 and inflation passage 114. The drainage passage and inlet passages have multiple openings. The multi-lumen tube has an expandable balloon such as expandable balloon 104 at its distal end. Thereafter at step 406, the tip of the catheter system distal to the expandable balloon inside the posterior urethra. At step 408, the openings on the drainage passage are adjusted to such that the openings face towards the anterior abdominal wall. At step 410, the expandable balloon is inflated by injecting a fluid such as distilled water through the inlet passage such as inlet passage 114. The inflated expandable balloon exerts pressure on the walls of the prostatic capsule and thus helps in controlling bleeding. Thereafter, at step 412, the urinary bladder walls and the abdominal walls are sutured. The catheter system may be removed after healing of the wounds in the urinary bladder.

The above described catheter system was used in approximately 96 patients which underwent suprapubic prostatectomy. The table 1 below provides a comparative analysis of the catheter system of the present invention and the existing catheter systems:

TABLE 1
Preopeartive and postoperative results in 96 patients undergoing suprapubic
prostatectomy with novel catheter v/s 50 with traditional technique

| | | Traditional | |
|---|---|---|---|
| Variables | Novel Catheter | Technique | p Value |
| No. pts | 96 | 50 | |
| Mean I-PSS ± SD (range): | | | |
| Preop | 31.6 ± 3.5 (21-35) | 31.6 ± 3.3 (24-35) | |
| Postop | 4.4 ± 2.2 (1-7) | 4.2 ± 2.1 (1-7) | |
| Mean ml/sec Qmax ± SD | | | |
| (range): | | | |
| Preop | 4.1 ± 2.8 (0-11 | 4.2 ± 2.4 (0-14) | |
| Postop | 22.6 ± 2.6 (14-25) | 22.5 ± 2.7 (15-25) | |
| Mean gm prostate wt ± SD | 63.3 ± 15.6 (20-110) | 64.3 ± 16.3 (55-85) | |
| (range) | | | |
| Mean cc balloon vol ± SD | 29.5 + 9 (10-50) | | |
| (range) | | | |
| Mean 24-hr drain | 155 ± 63.5 | 168.7 + 67.4 | 0.2 (independent |
| collection 1 ± SD (ml) | | | samples t test) |
| No. complications (%) | | | |
| Significant immediate or | — | 22 (44) | <0.001 (chi-square |
| late postop bladder | | | test) |
| irritative symptoms | | | |
| Clot retention | — | 19 (38) | <0.001 (chi-square |
| | | | test) |
| Bladder neck or urethral | 4 (4.1) | 7 (14) | 0.1 (chi-square test) |
| stricture | | | |
| Postop epididymo-orchitis | — | 4 (8) | <0.012 (Fischer's |
| | | | exact test) |
| Mean hemoglobin | 0.8 | 1.9 | <0.001 |
| decrease (mg/dl) | | | (independent |
| | | | samples t test) |
| No. catheter dislodgement | 2 (2.2) | — | |
| (%) | | | |
| No. early postop | 3 94) | 31 (62) | <0.001 (chi-square |
| incontinence (%) | | | test) |

I-PSS = International Prostatic Symptom Score;
Qmax = maximum urinary flow rate

The table clearly indicates that there was no report of clot retention or significant irritative symptoms in patients where the catheter system of the present invention was used.

The above described catheter system has a number of advantages. This urethral free catheter system is safe and effective. The use of the catheter system minimizes the post operative risk of infection, bladder irritation, incontinence and urethral stricture formation. Post-operatively, continuous irrigation can be obtained through the inlet and drainage passages of the multi-lumen tube of the catheter system.

While the preferred embodiments of the invention have been illustrated and described, it will be clear that the invention is not limited to these embodiments only. Numerous modifications, changes, variations, substitutions and equivalents will be apparent to those skilled in the art without departing from the spirit and scope of the invention as described in the claims.

What is claimed is:

1. A catheter system suitable for use in suprapubic prostatectomy, the catheter system comprising:
a multi-lumen tube having a plurality of lumens, the multi-lumen tube extending longitudinally for insertion into the urinary bladder, the multi-lumen tube comprising:
a plurality of outlet openings positioned on a first lumen, the plurality of outlet openings facilitating passage of fluid out of the urinary bladder;
at least one inlet opening positioned on a second lumen, the at least one inlet opening facilitating passage of fluid in to the urinary bladder; and
an expandable balloon, the expandable balloon being connected with the distal end of the multilumen tube, the expandable balloon positioned inside the prostate lodge
wherein the distal end of the catheter system is positioned in the urethra, the distal end of the catheter system being blocked to prevent leakage of fluid through urethra.

2. The catheter system according to claim 1 wherein the multi-lumen tube further comprises a third lumen, the third lumen being used for injecting a fluid in to the expandable balloon to inflate the expandable balloon.

3. The catheter system according to claim 2 wherein the injected fluid is distilled water

4. The catheter system according to claim 1 wherein the distal end is sealed by using a thread.

5. The catheter system according to claim 4 wherein the thread is made of 1-0 silk material.

6. The catheter system according to claim 1 wherein the plurality of outlet openings are positioned proximal to the expandable balloon.

7. The catheter system according to claim 1 wherein the at least one inlet opening is positioned in the area between the plurality of outlet openings.

8. The catheter system according to claim 1 wherein the cross section area of the plurality of outlet openings is between about 50 to 150 square millimeter.

9. The catheter system according to claim 1 wherein the diameter of the at least one inlet opening is between about three to seven millimeters.

10. The catheter system according to claim 1 wherein the distance between the plurality of outlet openings is not greater than about 10 millimeters.

11. The catheter system according to claim 1 wherein the expandable balloon is made of a biocompatible conductive plastic selected from at least one of polytetrafluroethylene (PTFE) and polyethylene.

12. The catheter system according to claim 1 wherein the plurality of outlet openings and the at least one inlet opening is circular in shape.
